(19) United States
(12) Patent Application Publication
(10) Pub. No.: US 2005/0118135 A1
(43) Pub. Date: Jun. 2, 2005

(54) System and methods for inducing ischemia in cells

(76) Inventor: Anthony C. Ross, John's Island, SC (US)

Correspondence Address: ATTN: LEGAL DEPARTMENT;NU VASIVE, INC., 4545 TOWNE CENTRE COURT, SAN DIEGO, CA, 92121, US

(21) Appl. No.: 10/975,267

(22) Filed: Oct. 28, 2004

Related U.S. Application Data
(60) Provisional application No. 60/515,248, filed on Oct. 28, 2003.

(51) Int. Cl. A61K 31/74

(52) U.S. Cl. 424/78.27; 424/78.31

(57) ABSTRACT

The present invention relates generally to inducing ischemia and, more particularly, to a method of inducing ischemia via the introduction of a flowable thermopolymer into a target vessel or structure which, after injection, will cool and solidify to obstruct or occlude the vessel or structure. This method of inducing ischemia is particularly suited for the treatment of cancer, by cutting off blood supply to a tumor to facilitate its removal, as well as removing organs as method of treatment or for transplant and/or draining and subsequent reduction and isolation of an organ for removal or for an organ to maintain its contents for removal.

### CROSS REFERENCES TO RELATED APPLICATIONS

The present application is a nonprovisional patent application claiming benefit under 35 U.S.C. § 119(e) from U.S. Provisional Application Ser. No. 60/515,248, filed on Oct. 28, 2003, the entire contents of which are hereby expressly incorporated by reference into this disclosure as if set forth fully herein.

### BACKGROUND OF THE INVENTION

I. Field of the Invention

The present invention relates generally to inducing ischemia and, more particularly, to a method of inducing ischemia via the introduction of a flowable thermopolymer into a target vessel or structure which, after injection, will cool and solidify to obstruct or occlude the vessel or structure. This method of inducing ischemia is particularly suited for the treatment of cancer, by cutting off blood supply to a tumor to facilitate its removal, as well as removing organs as method of treatment or for transplant and/or draining and subsequent reduction and isolation of an organ for removal or for an organ to maintain its contents for removal.

II. Discussion of the Prior Art

Cancer is a disease that claims the lives of millions of people worldwide. Although there are treatments for the disease, to date there is no cure. Most treatment options are incredibly unpleasant for the patient, and none of them can predict successful recovery with any degree of certainty. Furthermore, many treatment options have debilitating side effects that can affect the lives of cancer patients and their families for the rest of their lives.

All organs, limbs, and tumors rely on a nutrient supply to sustain life and maintain functionality. The greater the metabolic activity of a structure, the more nutrients are required to remain viable. Active or highly aggressive tumors require a rich blood supply in order for the tumor to proliferate.

Much of the contemporary treatment of cancer addresses the cellular production or blood supply. Presently the most-utilized treatments for cancer include surgery, radiation, and chemotherapy. In addition, many alternative or experimental treatments are also available, including immunotherapy, hyperthermia, radio frequency ablation, hormonal therapy, angiogenesis inhibitors, photodynamic therapy, and vaccine therapy.

Each of the primary cancer treatments is highly invasive to the human body, but each in a different way. Removal of tumors via surgery is the most physically invasive technique, yet it potentially carries the lowest degree of damage from side effects. In this treatment, the tumor is physically removed from the body through mechanical processes. Surgical removal can be effective for small tumors, but often it is not possible to extract the entire tumor, and the end result is that cancer remains in the patient. Even if the entire tumor can be removed, doctors must also remove significant portions of healthy tissue and lymph nodes along with the tumor, causing damage to the body.

Radiation therapy, or radiotherapy, is a treatment method in which high-energy rays are used to treat the cancer cells. Radiation is often used in conjunction with surgical treatment, either pre-surgery to shrink the tumor to allow for easier removal, or post-surgery to remove any remaining cancer cells from the tissue. Radiotherapy does have serious, though mostly temporary, side effects. For instance, patients may experience fatigue, hair loss, skin discoloration, and a decrease in infection-fighting white blood cells.

The third conventional method of treating cancer is chemotherapy, in which anticancer drugs are administered in a number of ways, including intravenously through a catheter, intrathecally (into the cerebral spinal fluid) through a needle placed in the spine, or by a heavy dosage of pills. The chemicals once introduced are intended to kill the cancer cells. However, chemotherapy also has side effects that are very similar to, if not more severe than, radiotherapy. For example, patients may experience fatigue, hair loss, nausea, vomiting, and general painful discomfort. More serious side effects include permanent infertility and cessation of the menstrual cycle in women. Unlike radiation, chemotherapy is systemic in nature and therefore exposes the entire body to chemicals, increasing the danger of side effects.

In order to increase the effectiveness and decrease the discomfort associated with the conventional treatments discussed above, many alternative and experimental treatments have emerged in recent years. One such treatment is immunotherapy, in which the body's immune response is increased in order to fight disease or protect from the harmful side effects of other treatments. Some types of immunotherapy include herbal remedies, monoclonal antibodies, interferons, interleukin-2, and colony stimulating factors. Another type of immunotherapy is the use of cancer vaccines. Ordinarily vaccines are used as preventative treatment, administered prior to contraction of a disease in order to prepare the immune system to rapidly fight the disease. However, cancer vaccines are therapeutic in nature: instead of preparing the body to fight future cancer, they aid the body's immune system in fighting existing cancer. Immunotherapy as a primary treatment for cancer itself if ineffective at best, and as a result it is used primarily a means to combat side effects of more conventional treatments.

Another method of treating cancer is hyperthermia, or heating the malignancy to its lethal capacity, heat-killing the tumor. The idea is that since cancer cells have a higher temperature than normal healthy cells, they will reach the lethal temperature for cells faster than the surrounding tissue. However, to date the conventional methods of heating are inadequate because they primarily use external sources of heat and thus cannot reach deep-rooted tumors without creating a toxic temperature gradient within the healthy tissue surrounding the cancer.

An experimental derivative of the hyperthermia treatment currently in development is Intracellular Hyperthermia Therapy (ICHT). The idea behind ICHT is that if the cancer cells could be heated from the inside, then the damage to surrounding tissue from hyperthermia would be minimized. The suggested method is to introduce an “uncoupling agent” into the bloodstream, which would then infiltrate cells and boost their metabolism on the order of four-fold. Cancer cells, with a much higher metabolism, would be pushed beyond their lethal limit, while normal cells would in theory be unaffected. However, the problem with this treatment is that it still has some effect on normal cells. Specifically, the effect of increased metabolism on normal cells is unknown, and may turn out to be harmful.

Another relatively new cancer treatment is radio frequency ablation (RFA), in which RF energy is deposited directly into the tumor by a probe. The energy heats the cells beyond their fatal temperature, destroying them. Although still experimental in nature and primarily used for treating liver cancer, this treatment is not without problems. First, the treatment can be extremely painful for the patient, and various forms of sedation are recommended, ranging from conscious sedation to general anesthesia. Secondly, in order to increase the likelihood of success, a zone of normal cells surrounding the tumor must also be killed. Thirdly, tumors located near major arteries cannot be killed completely since the arteries will siphon some of the heat away from the tumor.

Hormone therapy is an alternative treatment in which cancer cells are starved of the hormones that they need to grow. This can be accomplished either through the administration of hormone-suppressing drugs or surgery to remove a hormone-producing organ. In addition to side effects that are similar to chemotherapy, hormone therapy is only effective against certain types of cancers.

The use of angiogenesis inhibitors is currently under investigation. The idea behind this type of treatment is to arrest the formation of new blood vessels that provide nutrient-rich blood to the tumors. If such formation can be stopped, then the cancer cells will essentially die from malnutrition. However, the primary drawback to this treatment is that it is still highly experimental, and the effectiveness in fighting cancer is not yet known.

Yet another type of alternative cancer treatment is photodynamic therapy (also called PDT, phototherapy, photochemotherapy, or photoradiation therapy). PDT is based on the use of photosensitizing agents that can kill one-celled organisms when exposed to a certain type of light. In this treatment, a photosensitizing agent is introduced into the bloodstream and absorbed by cells all over the body, including the target tumor. Since the agent will remain in cancer cells longer than in healthy cells, treatment is delayed for a period of time after introduction of the agent. The tumor is then treated with a laser, activating the photosensitizing agent and causing the production of an active form of oxygen, which will kill the cells. However, many problems are associated with this treatment. First, there is the aforementioned timing problem, and more specifically picking the right time to be most effective. Secondly, the patient's eyes and skin remain ultra-sensitive to light for at least a period of six weeks. Finally, since the lasers used cannot pass through more than three centimeters of tissue, PDT is not effective against deeper seeded tumors.

In addition to the specific problems with each treatment discussed above, the central disadvantage to many of these cancer treatments is that it is difficult to isolate the tumors and apply the treatment method only to the target area. As a result, many healthy cells are destroyed along with the tumor. If the treatment is systemic in nature, this can cause a multiplicity of problems all over the body. Similarly, localized treatments can damage significant portions of the body surrounding the tumor.

Furthermore, treatments involving introduction of foreign elements not accepted by the body—such as chemicals, radiation, or excessive heat—can cause uncomfortable, painful, or even debilitating side effects that may be more than temporary in nature.

The present invention is directed at overcoming, or at least improving upon, the disadvantages of the prior art.

### SUMMARY OF THE INVENTION

The present invention accomplishes the above-identified problems by providing a method of treating cancer using a thermopolymer composition to block arterioles feeding the tumor and induce ischemia, thereby killing the cancer cells. Prior to injection, the thermopolymer material is heated to a temperature sufficient to create a flowable form, whereby it may then be injected into the body. After injection into the arterioles, the material will cool to body temperature and solidify, creating an artificial barrier.

According to one broad aspect of the present invention, this method of treating cancer comprises a nonreactive thermopolymer composition capable of injection in flowable form and an injection apparatus. It is contemplated that the injection apparatus may itself be able to heat the thermopolymer to flowable form.

The thermopolymer composition contains the thermopolymer matrix and a dispersion compound. The thermopolymer matrix may include any number of suitable materials capable of being heated to flowable form and injected into an artery, filling the artery and solidifying upon cooling to body temperature in order to form a barrier. The thermopolymer must not react negatively with the body. By way of example only, the thermopolymer matrix may include bone wax, paraffin, gutta percha, balata, polyisoprene and/or any mixture of bone wax, paraffin, gutta percha, balata and/or polyisoprene.

The dispersion compound may include any number of compositions having suitable mechanical, chemical, radiopacity, anti-microbial and/or anti-inflammatory characteristics. By way of example only, the dispersion compound may include, but is not necessarily limited to, titanium, crystalline particles, gold (in any form) and/or and combination of titanium, crystalline particles, and/or gold.

The injection device may include any number of mechanisms capable of injecting molten thermopolymer into the body. By way of example only, the injection device may include an injection gun or simple syringe. In a preferred embodiment, the injection gun would be capable of heating the thermopolymer to the desired temperature in order to facilitate dispersion in a molten form. The preferred injection gun would also be able to maintain the temperature of the thermopolymer composition so as to maintain molten consistency throughout application. The preferred embodiment would also contain a specialized injection needle to facilitate optimal dispersion of the thermopolymer compound. In an alternative embodiment, the thermopolymer composition would be heated using an independent device such as a hot pot, open flame, or microwave, and then transferred to a syringe for injection.

### BRIEF DESCRIPTION OF THE DRAWINGS

Many advantages of the present invention will be apparent to those skilled in the art with a reading of this specification in conjunction with the attached drawings, wherein like reference numerals are applied to like elements and wherein:

FIG. 1 is a flow chart of a method of treating cancer according to an exemplary embodiment of present invention;

FIG. 2 illustrates the in vivo mechanics of the method of the present invention;

FIG. 3 illustrates an exemplary embodiment of an injection gun suitable for use in injecting a thermopolymer according to the present invention;

FIG. 4 illustrates a cannula for use in an exemplary embodiment of an intravascular injection system for use in injecting a thermopolymer according to the present invention;

FIGS. 5-7 illustrate an enlarged view of the distal end of a cannula for use in an exemplary embodiment of an intravascular injection system for use in injecting a thermopolymer according to the present invention; and

FIG. 8 illustrates an exemplary embodiment of an endoscopic delivery system for use in injecting a thermopolymer according to the present invention.

### DESCRIPTION OF THE PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decision must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The method of inducing ischemia according to the present invention will be discussed in detail below with respect to its exemplary utility in treating cancer. However, it will be appreciated by those skilled in the art (and is within the scope of the present invention) that the methodology of the present invention may also find use in removing organs as method of treatment or for transplant and/or draining and subsequent reduction and isolation of an organ for removal or for an organ to maintain its contents for removal. The method of inducing ischemia disclosed herein boasts a variety of inventive features and components that warrant patent protection, both individually and in combination.

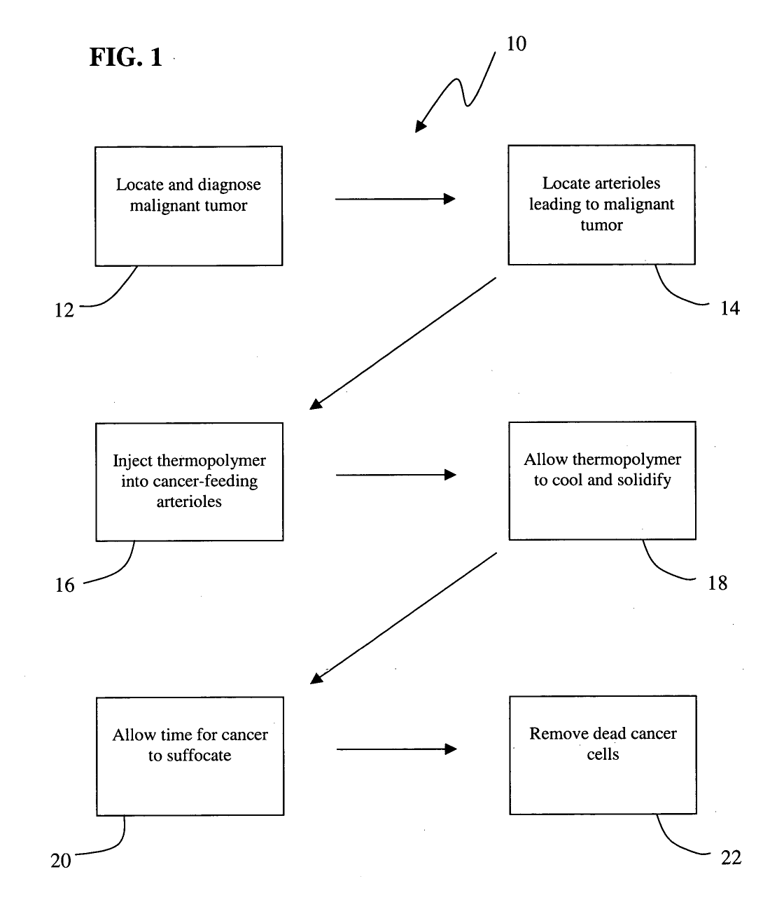

FIG. 1 is a flowchart illustrating the major steps of a method 10 of treating cancer according to an exemplary embodiment of the present invention. The first step 12 is to locate and diagnose the malignant tumor. This may be accomplished in any number of suitable fashions (currently existing or later developed), including but not limited to the use of Magnetic Resonance Imaging (MRI), X-ray imaging, ultrasound, and/or physical inspection. The second step 14 is to locate each arteriole supplying blood to the tumor. This may be accomplished in any number of suitable fashions (currently existing or later developed), including but not limited to the use of MRI, X-ray imaging, ultrasound, and/or physical inspection. The next step 16 is to inject a flowable thermopolymer into the arterioles leading to cells in and around the tumor. This can be accomplished in any number of suitable fashions (currently existing or later developed), including but not limited to needle injection, intravascular delivery (e.g. catheter based), and/or endoscopic devices. After the thermopolymer cools and solidifies 18, it will act as a dam to block the arteriole and close off the blood supply to the tumor. In time 20, the tumor will in effect suffocate and die. At this point 22, the dead cell mass may be surgically removed if desired.

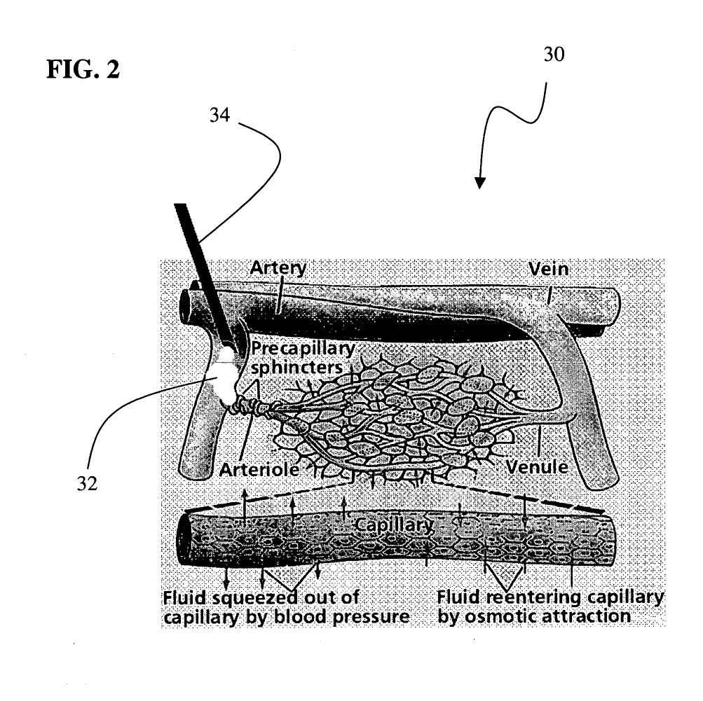

FIG. 2 is a schematic illustrating the thermopolymer injection step. In this diagram 30 one can see that the nutrient-rich blood flows from the arteries through arterioles to capillaries that feed cells. Preventing the blood from flowing through the arterioles will divert nutrients from the tumor, and the cells will starve to death. To achieve this, the thermopolymer 32 is injected through an injection needle 34 such that it enters the arterioles leading to cells in and around the tumor. Upon cooling, the thermopolymer 32 will form internal dams within the arterioles that will block the arterioles and divert the blood flow from the tumor.

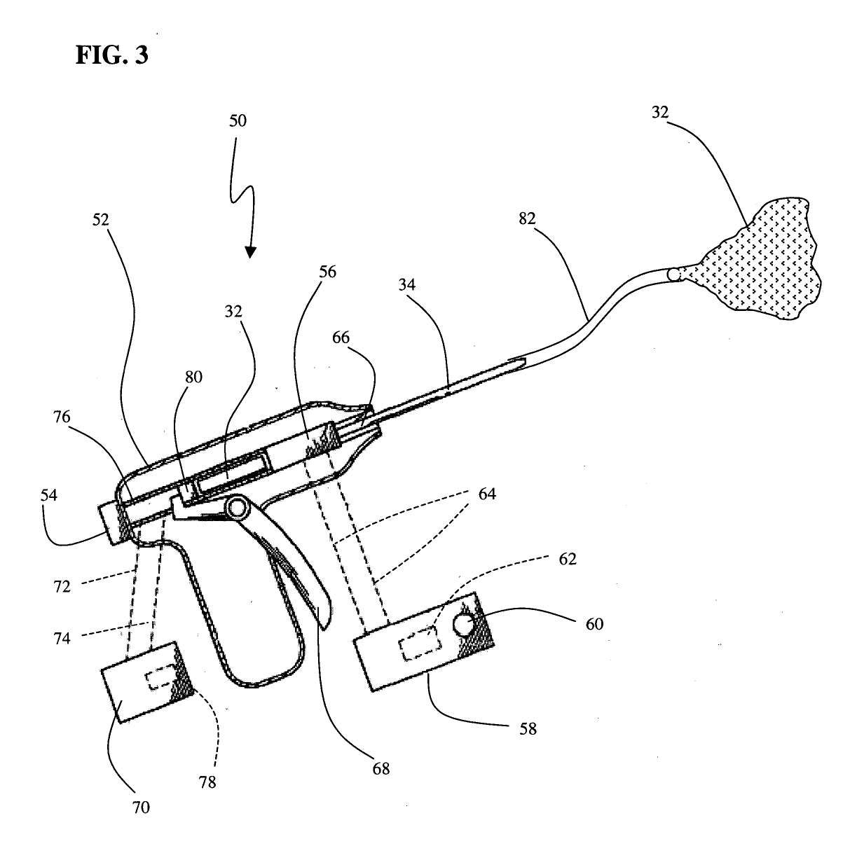

FIG. 3 illustrates an exemplary embodiment of an injection gun 50 suitable for use in inserting a thermopolymer 32 according to the present invention. Specifically, injection of thermopolymer 32 through cannula 82 by injection gun 50 is shown. Injection gun 50 has a body 52 with a removable plunger 54 adapted to receive a cylindrical plug of the thermopolymer material 32. A heater 56 may be provided to heat thermopolymer material 32 and a heater control unit 58 having an adjustable temperature control knob 60 may be provided with a temperature readout at 62. Electrical leads 64 extend to heater 56. An injection needle 34 extends from body 52 and has a ceramic sheath 66 about a portion of the proximal end of needle 34. Cannula 82 may be attached to distal end of needle 34 to facilitate injection into the body. Injection needle 34 may be composed of any number of suitable materials, including but not limited to silver, aluminum, or stainless steel. A hand-operated trigger 68 may be activated for forcing thermopolymer material 32 from the end of needle 34 into cannula 82 upon heating of the thermopolymer material to a predetermined temperature. To assist trigger 68 in exerting an axial force against the plug of thermopolymer 32 in gun 50, a foot operated hydraulic pump 70 may be provided to supply fluid through lines 72, 74 to hydraulic cylinder 76. A pressure readout is provided at 78. A suitable piston 80 may exert an axial force against the thermopolymer material 32. A hydraulic system is effective in providing an axial injection force that may be easily regulated and controlled by personnel performing the procedure.

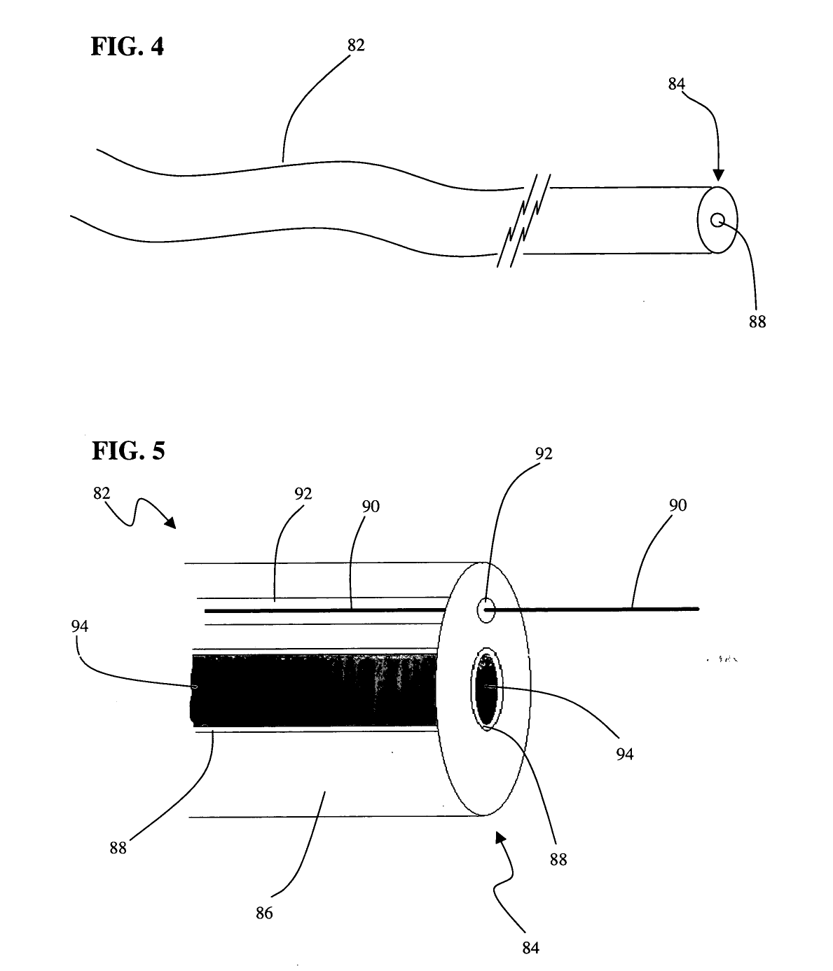

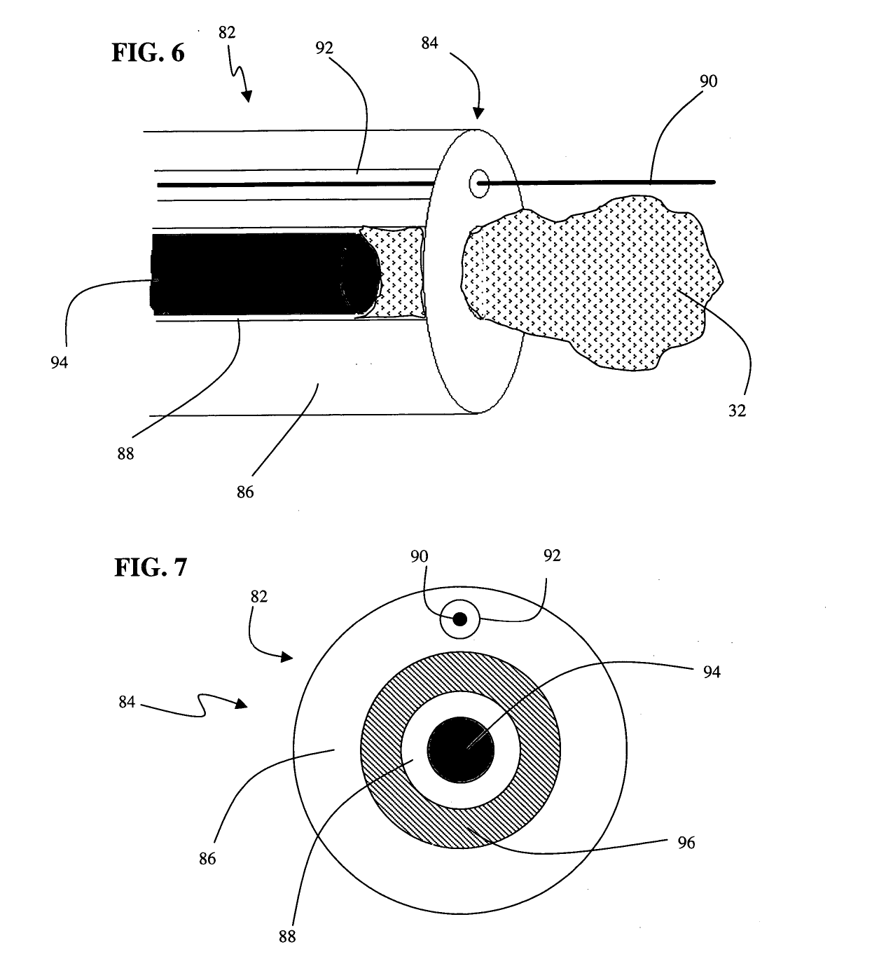

FIGS. 4-7 illustrate an exemplary embodiment of an intravascular injection system for use in inserting a thermopolymer according to the present invention. FIG. 4 shows the main body and distal end 84 of cannula 82. Located at distal end 84 of cannula 82 is the distal opening of inner lumen 88. FIG. 5 represents an enlarged side prospective view of distal end 84 of cannula 82. Inner lumen 88 contains retractable plunger 94, shown in the closed position. Inner lumen 88 extends in a generally central manner along the length of cannula 82 and is attached to the distal end of injection needle 34 of injection gun 50 of FIG. 3 to permit flowage of thermopolymer 32. Surrounding inner lumen 88 is catheter body 86. Catheter body 86 may be comprised of any material capable of providing a solid yet flexible and insular housing for inner lumen 88, including but not limited to rubber, plastic, latex, or silicon. Guide wire 90 extends the length of cannula 82 through guide wire lumen 92. Guide wire lumen 92 is generally a smaller tube than inner lumen 88, and is located in a generally superior orientation to inner lumen 88 within catheter body 86.

FIG. 6 illustrates the distal end 84 of cannula 82 with retractable plunger 94 in the open position. Retraction of plunger 94 allows for flowage of thermopolymer 32 into the targeted arteriole. FIG. 7 is a frontal view of the distal end 84 of cannula 82, illustrating the general orientation of the components. Inner lumen 88 is oriented in a generally central location of cannula 82. Retractable plunger 94 is located in the interior of inner lumen 88. Heating element 96 may surround inner lumen 88 in order to facilitate heating (or prevent premature cooling) of thermopolymer 32. Heating element 96 may comprise any number of suitable elements for heating, including but not limited to ceramics, coils, and the like. Catheter body 86 surrounds inner lumen 88 and, if present, heating element 96. Guide wire 90 is located inside guide wire lumen 92, which extends through catheter body 86 and is generally located in a superior position to inner lumen 88 and heating element 96 (if present).

Guide wire 90 can be inserted into the body by any of a number of well-known methods, such as the Seldinger technique. Once guide wire 90 is placed, cannula 82 is advanced by introducing the proximal end of guide wire 90 into guide wire lumen 92 located at distal end 84 of cannula 82. Cannula 82 is then passed along guide wire 90 until the desired location is reached within the body. Once cannula 82 is properly inserted, trigger 68 of insertion gun 50 is activated, forcing heated thermopolymer 32 through needle 34, into cannula 82, and eventually into the proper arteriole.

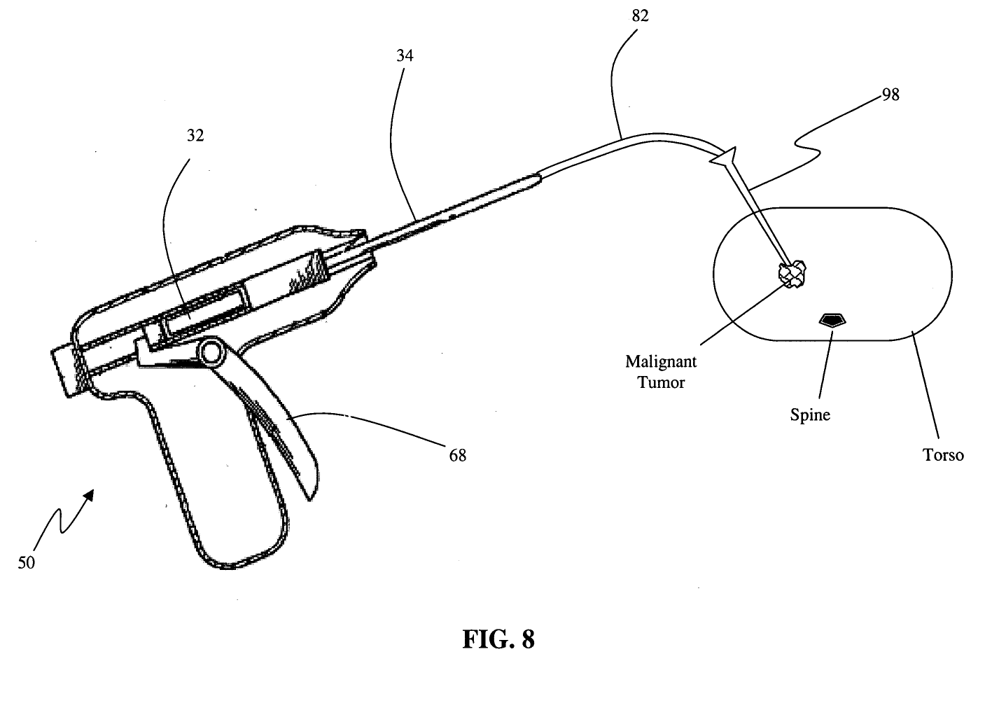

FIG. 8 illustrates an exemplary embodiment of an endoscopic delivery system for use in injecting a thermopolymer according to the present invention. Once a malignant tumor is located in the patient (shown here in the torso, but could be anywhere), the method 10 of treating cancer may be utilized. A trocar 98, or any other device commonly used by those skilled in the art to insert intravenous cannulae, may be used to facilitate insertion of cannula 82 into the proper arteriole. Once cannula 82 is properly inserted, trigger 68 of insertion gun 50 is activated, forcing heated thermopolymer 32 through needle 34, into cannula 82, and eventually into the proper arteriole. Once thermopolymer 32 has been inserted, only a short time is needed to allow for solidification and consequential blockage of the tumor feeding arteriole.

While the present invention has been shown and described in terms of preferred embodiments thereof, it should be understood that this invention is not limited to any particular embodiment, and that changes and modifications may be made without departing from the true spirit and scope of the invention as defined in the appended claims. By way of example only, the method of inducing ischemia according to the present invention may also find use in removing organs as method of treatment or for transplant. This may be accomplished via oblation or occluding the blood flow, which facilitates the removal of the target organ by restricting blood flow within the organ prior to removal and hence blood loss during removal. In addition, the method of inducing ischemia of the present invention may also find use in maintaining the contents of the target organ during removal, such as by occluding the egress portal ( [symbol] ) of the target organ via injecting a thermopolymer according to the present invention (as described above with respect to blood flow obstruction).

1. A method for inducing ischemia in cells, comprising:
injecting a thermopolymer composition into at least one arteriole delivering blood to at least one target cell; and
allowing sufficient time for said at least one target cell to die.

2. The method of claim 1, wherein said thermopolymer composition comprises a heated thermopolymer composition.

3. The method of claim 1, wherein said thermopolymer composition comprises a thermopolymer matrix and a dispersion compound.

4. The method of claim 3, wherein said thermopolymer matrix is composed of at least one of bone wax, paraffin, gutta percha, balata, and polyisoprene.

5. The method of claim 3, wherein said dispersion compound is composed of at least one of titanium, crystalline particles, and gold.

6. The method of claim 1, wherein said at least one target cell comprises a malignant cancer cell.

7. The method of claim 2, further comprising:
allowing said thermopolymer composition sufficient time to cool.

8. A method for treating cancer, comprising:
locating at least one malignant tumor cell;
identifying at least one arteriole delivering blood to said at least one malignant tumor cell;
injecting a thermopolymer composition into said at least one arteriole; and
allowing sufficient time for said at least one malignant tumor cell to die.

9. The method of claim 8, wherein said thermopolymer compound comprises a heated thermopolymer compound.

10. The method of claim 8, wherein said thermopolymer composition comprises a thermopolymer matrix and a dispersion compound.

11. The method of claim 10, wherein said thermopolymer matrix is composed of at least one of bone wax, paraffin, gutta percha, balata, and polyisoprene.

12. The method of claim 10, wherein said dispersion compound is composed of at least one of titanium, crystalline particles, and gold.

13. The method of claim 9, further comprising:
allowing sufficient time for said heated thermopolymer composition to cool.

14. The method of claim 8, further comprising:
removing said dead malignant tumor cells from the body.

15. A system for inducing ischemia in a cell, comprising:
a thermopolymer composition for injecting into at least one blood vessel; and
an injection apparatus for injecting said thermopolymer composition into said blood vessel.

16. The system of claim 15, wherein said thermopolymer composition comprises a heated thermopolymer composition.

17. The system of claim 15, wherein said thermopolymer composition comprises a thermopolymer matrix and a dispersion compound.

18. The system of claim 17, wherein said thermopolymer matrix is composed of at least one of bone wax, paraffin, gutta percha, balata, and polyisoprene.

19. The system of claim 17, wherein said dispersion compound is composed of at least one of titanium, crystalline particles, and gold.

20. The system of claim 15, wherein said injection apparatus comprises at least one of an injection gun and simple syringe.

21. The system of claim 20, wherein said injection gun comprises:
a chamber for receiving said thermopolymer in a non-flowing state;
a heating element for heating said thermopolymer to a flowing state;
a hollow injection needle to receive the flowing thermopolymer for injection into said blood vessel; and
a force applying assembly to apply force against said thermopolymer to force the thermopolymer from said needle when heated to a flowing state.

22. The system of claim 21, further comprising:
a plug of said thermopolymer within said chamber, said force applying assembly including a piston adjacent said plug for forcing said thermopolymer from said needle after said thermopolymer is heated to a flowing state.

23. The system of claim 22, wherein said force applying assembly comprises a hydraulic force applying assembly for hydraulically actuating said piston.

24. The system of claim 23, further comprising:
a fluid pressure source for said hydraulically actuated piston; and
a foot operated pedal associated with said fluid pressure source to provide a selected fluid pressure to said piston and heated thermoplastic material.

25. The system of claim 15, further comprising:
a heater for heating said thermopolymer to a flowable form prior to injection into said blood vessel.

26. The system of claim 15, further comprising:
a cannula attached to said injection apparatus to facilitate injection of said thermopolymer into said blood vessel.

27. The system of claim 26, wherein said cannula comprises:
an inner lumen extending in a generally central manner along the length of said cannula, said inner lumen providing a conduit for flowage of said thermopolymer from said injection apparatus into said blood vessel; and
a flexible catheter body surrounding said inner lumen.

28. The system of claim 27, further comprising:
a retractable plunger situated in the interior of said inner lumen.

29. The system of claim 27, further comprising:
a guide wire lumen extending though the length of said flexible catheter body generally adjacent to said inner lumen; and
a guide wire extending at least the length of said cannula through said guide wire lumen.

30. The system of claim 29, wherein said guide wire lumen is located in a generally superior orientation relative to said inner lumen.

31. The system of claim 27, further comprising:
a heating element surrounding said inner lumen for facilitating heating and preventing premature cooling of said thermopolymer.

32. The system of claim 31, wherein said heating element comprises at least one of ceramics and coils.

33. A method of inducing ischemia in cells, comprising:
heating a thermopolymer composition to a flowable form;
transferring said heated thermopolymer composition to an injection device;
injecting said thermopolymer composition into at least one arteriole delivering blood to at least one target cell; and
allowing sufficient time for said at least one target cell to die.

34. The method of claim 33, wherein heating a thermopolymer composition to a flowable form comprises using at least one of a hot pot, open flame, and microwave to heat said thermopolyer to a flowable form.

35. The method of claim 33, wherein said injection device comprises at least one of an injection gun and simple syringe.
